(19) United States
(12) Patent Application Publication
(10) Pub. No.: US 2008/0262097 A1
(43) Pub. Date: Oct. 23, 2008

(54) Formulations

(76) Inventors: Elizabeth Anne Eady, West Yorkshire (GB); Jonathan Howard Cove, West Yorkshire (GB); Daniel James Fitzgerald, Leeds (GB); Scott Seville, West Yorkshire (GB)

Correspondence Address: BEYER WEAVER LLP, P.O. BOX 70250, OAKLAND, CA, 94612-0250, US

(21) Appl. No.: 11/909,066

(22) PCT Filed: Mar. 23, 2006
(86) PCT No.: PCT/GB06/01076

§ 371 Date: Jun. 30, 2008

(30) Foreign Application Priority Data
Mar. 23, 2005 (GB) 0505909.2

(51) Int. Cl. A61K 31/075; A01N 31/06; A01P 1/00; A61P 31/04

(52) U.S. Cl. 514/714

(57) ABSTRACT

An antimicrobial formulation containing (a) a peroxide selected from the group consisting of diacyl peroxides, alkyl hydroperoxides and metal peroxides and (b) a benzoquinone or hydroquinone. The formulation may in particular be used against staphylococci or propionibacteria, more particularly to treat skin and skin structure conditions such as acne.

### FIELD OF THE INVENTION

This invention relates to antimicrobial formulations, and to the use of certain combinations of compounds as antimicrobial agents.

### BACKGROUND TO THE INVENTION

Peroxides such as benzoyl peroxide are known for use in topical formulations for treating skin infections, most notably for the treatment of acne. Benzoyl peroxide in particular is well known for its keratolytic (comedolytic) activity.

However peroxides are also known to act as skin irritants and as bleaching and oxidising agents. These side effects limit the concentrations in which peroxides can be used in topical skin preparations, and are naturally off-putting to users.

It has now surprisingly been found that when certain types of peroxide are combined with certain types of quinone, a synergistic effect can be observed on their combined level of antimicrobial activity. As a result, novel antimicrobial formulations can be prepared, in particular for topical application, either with improved efficacy and/or containing lower levels of peroxide actives compared to previous such formulations.

### STATEMENTS OF THE INVENTION

According to a first aspect of the present invention there is provided an antimicrobial formulation containing (a) a peroxide selected from the group consisting of diacyl peroxides, alkyl hydroperoxides and metal peroxides and (b) a benzoquinone or hydroquinone.

This formulation is preferably suitable for topical application to, and/or contact with, the skin, in particular human skin. The peroxide and the benzo/hydroquinone are therefore preferably contained in a pharmaceutically acceptable vehicle which can safely be applied to, and/or contacted with, the skin and/or other epithelia. Ideally the formulation is suitable for topical application to areas such as the nares, eyes, scalp and/or vagina, and/or to tissue areas within the ears and/or the oral cavity. Suitability for application to the skin, nares and tissue within the ears is most preferred, in particular the skin and nares.

A formulation which is “suitable for” topical application may also be adapted for topical application.

Suitable vehicles will be well known to those skilled in the art of preparing topical skin care or pharmaceutical preparations. The vehicle will typically be a fluid, which term includes a cream, paste, gel, lotion, ointment, foam or other viscous or semi-viscous fluid, as well as less viscous fluids such as might be used in sprays (for example for nasal use). The peroxide and the benzo/hydroquinone may each independently be present in the form of a solution or suspension, the term “suspension” including emulsions and other multi-phase dispersions.

Either or both of the peroxide and the benzo/hydroquinone may, whether separately or together, be carried in or on a delivery vehicle which is suitable for targeting or controlling its release at the intended site of administration. Such vehicles include liposomes and other encapsulating entities, for example niosomes, aspasomes, microsponges, microemulsions, hydrogels and solid lipid nanoparticles.

A peroxide is a compound containing the O₂²⁻ group. It may be inorganic or organic, although it will not typically be hydrogen peroxide (H₂O₂). According to the present invention, the peroxide is selected from diacyl peroxides (eg, diacetyl peroxide, didecanoyl peroxide, dilauroyl peroxide or dibenzoyl peroxide), alkyl hydroperoxides (eg, t-butyl hydroperoxide) and metal peroxides. Generally speaking, diacyl peroxides may be preferred.

Suitable metal peroxides include zinc, calcium, lithium, sodium, barium, magnesium and copper peroxides. Most preferred are group (I) or group (II) metal peroxides, particularly the latter—examples include sodium, magnesium and calcium peroxides, of which calcium and magnesium peroxides, especially calcium, are preferred.

Diacyl peroxides have the formula R¹—C(O)—OO—C(O)—R², where R¹ and R² are each independently selected from alkyl, cycloalkyl and aryl groups, preferably aryl. Suitable alkyl groups may be selected from C₁ to C₂₀ groups, preferably from C₁ to C₁₆ groups, more preferably from C₁ to C₁₂ groups, most preferably from C₁ to C₈ or C₁ to C₆ or C₁ to C₄ (for example methyl, ethyl, propyl (preferably iso-propyl) or butyl (preferably t-butyl)) groups. A suitable aryl group is phenyl.

A diacyl peroxide may in particular be derived from a fatty acid source; for example it may be a dilauryl, diamyl or dicumyl peroxide. An alkyl, cycloalkyl or aryl group may be substituted with one or more other groups, but is preferably unsubstituted.

Suitably R¹ and R² are each independently selected from C₁ to C₁₂ alkyl and C₁ to C₈ aryl, in particular phenyl.

Some suitable diacyl peroxides, of medium chain (C₆ to C₁₂) fatty acids, are disclosed for instance in U.S. Pat. No. 4,479,939. More preferably at least one of R¹ and R² (preferably both) is phenyl.

Alkyl hydroperoxides have the formula R³—OO—H where R³ is selected from alkyl, cycloalkyl and aryl groups, preferably alkyl, preferably C₁ to C₆ or C₁ to C₆ or C₁ to C₄ groups. In general R³ may be as defined above for R¹ and R². Most preferably R³ is t-butyl.

It may be preferred for the peroxide to be selected from diacyl peroxides and metal peroxides.

In particular where the formulation is for use against staphylococci, it may be preferred for the peroxide to be a diacyl peroxide, preferably benzoyl peroxide (also known as dibenzoyl peroxide or benzoyl superoxide), which has the formula Ph-C(O)—O—O—C(O)-Ph.

In particular where the formulation is for use against propionibacteria, it may be preferred for the peroxide to be selected from diacyl peroxides (preferably benzoyl peroxide), alkyl hydroperoxides (preferably t-butyl hydroperoxide) and metal peroxides (preferably calcium, magnesium or sodium peroxide, more preferably calcium or magnesium peroxide).

The peroxide may in particular be benzoyl peroxide.

A formulation according to the invention may contain more than one peroxide.

A benzoquinone is a cyclohexadiene-dione, containing two C═O groups in an unsaturated 6-membered ring. The four remaining carbon atoms may carry one or more substituents—in other words, the benzoquinone may be optionally substituted. The term “benzoquinone” is not however intended to embrace bi- or poly-cyclic quinones.

A hydroquinone (sometimes known as a hydroxyquinone) is a benzoquinone in which one or more—typically both—of the C═O groups is instead present as a C—OH group; in other words, it is typically a dihydroxy benzene, optionally substituted with one or more additional groups.

A benzoquinone may be present at least partly in the form of the corresponding hydroquinone, or vice versa, or either may exist at least partly as a radical in which one or more of the C═O or C—OH groups is present as C—O.. Such compounds may, depending in part on their local environment (for example pH), be present in the form of an equilibrium mixture of two or more such species, for instance of the benzoquinone and its corresponding hydroquinone. At alkaline pHs, for example, the compounds are more likely to be present in the form of the benzoquinones, whereas at acidic pHs they are more likely to be present as the hydroquinones. The presence of an oxidising agent, such as a peroxide, may also induce at least partial conversion of a hydroquinone to the corresponding benzoquinone. The present invention thus embraces the use of a benzoquinone, a hydroquinone, a corresponding radical or any mixture of two or more such species.

The two C═O groups or C—OH groups of a benzo/hydroquinone may be positioned ortho, meta or para to one another. When positioned ortho to one another, this is known as a cyclohexadiene-1,2-dione or o-benzoquinone or, in the case of the corresponding hydroquinone, a catechol. When positioned meta to one another, this is known as a cyclohexadiene-1,3-dione or an m-benzoquinone or, in the case of the corresponding hydroquinone, a resorcinol. When positioned para to one another, this is known as a cyclohexadiene-1,4-dione or a p-benzoquinone or, in the case of the para-substituted HO-Ph-OH, simply as a p-hydroquinone.

Preferably the two C═O groups or C—OH groups are positioned ortho or para to one another, most preferably para.

The benzo/hydroquinone used in the invention may be, and in cases is preferably, substituted with one or more other groups such as those selected from alkyl, alkoxy, halogen, hydroxyl, nitro (—NO₂) and amine (—NR₂, where each R is independently either hydrogen or hydrocarbyl) groups. Such groups will be attached to carbon atoms in the cyclohexadiene ring of the quinone.

In general, an alkyl substituent may be either a straight or a branched chain alkyl group. It may be or contain cycloalkyl moieties. It may contain for instance from 1 to 12 carbon atoms, preferably from 1 to 10, more preferably from 1 to 8. An alkyl substituent is preferably a C₁ to C₆ alkyl group, more preferably a C₁ to C₅ or C₁ to C₄ alkyl group. Secondary and tertiary alkyl groups (for example iso-propyl and t-butyl) may be preferred. Suitable alkyl groups may therefore be selected from methyl, ethyl, iso-propyl and t-butyl.

An alkoxy substituent is preferably a C₁ to C₆ alkoxy group, more preferably a C₁ to C₅ or C₁ to C₄ or C₁ to C₂ alkoxy group, most preferably methoxy. A halogen substituent may be selected from fluorine, chlorine and bromine, preferably fluorine or chlorine, most preferably chlorine. An amine substituent is preferably NH₂.

The benzo/hydroquinone is preferably substituted with at least one such substituent, which is preferably (at least in the case of a meta- or para-substituted benzo/hydroquinone) at the 2-position or (in the case of an ortho-substituted compound) at the 3-position. In some cases the benzo/hydroquinone may be substituted with two such substituents, in other cases with three or even four. It may be preferred for the benzo/hydroquinone to have either one or two such substituents, in some cases just one.

Particularly preferred are substituents selected from alkyl, alkoxy, halogen and nitro groups, or from alkyl, alkoxy and halogen groups, or from alkyl and halogen groups, or from alkyl and alkoxy groups. Most preferred substituents are the alkyl groups, in particular C₁ to C₄ alkyl groups.

The benzo/hydroquinone may be substituted with up to four alkyl groups, but in particular may be a mono- or di-alkyl benzo/hydroquinone.

The benzo/hydroquinone may for instance be substituted with one butyl group, which is preferably present at the 2-position; it may however be substituted with more than one butyl group, for instance two. A butyl group is preferably a t-butyl group.

The benzo/hydroquinone may be substituted with two butyl groups. These may for instance occupy the 2- and 5-positions, in particular where the benzo/hydroquinone is a para-benzo/hydroquinone. They may alternatively occupy the 3- and 5-positions, in particular where the benzo/hydroquinone is an ortho-benzo/hydroquinone. Again the butyl groups are preferably t-butyl groups.

Instead or in addition, the benzo/hydroquinone may be substituted with one methyl group, which is preferably present at the 2- or the 5-position; it may however be substituted with more than one methyl group, for instance two or three or even four. It may for instance be substituted with two methyl groups, which are preferably present at the 2- and 3-positions. It may be substituted with three methyl groups, which are preferably present at the 2-, 3- and 5-positions.

Instead or in addition, the benzo/hydroquinone may be substituted with one propyl group, which is preferably present at the 2-position. The benzo/hydroquinone may however be substituted with more than one propyl group, for instance two. A propyl group is preferably an iso-propyl group.

Instead or in addition, the benzo/hydroquinone may be substituted with one, two, three or even four ethyl groups, preferably one or two, more preferably one and yet more preferably at least one of them occupying the 2-position.

Although in many cases this will not be preferred, a hydroquinone may instead or in addition carry one or two (preferably one) substituents attached directly to the oxygen atom(s) of its C—OH groups (thus replacing the hydrogen atom(s) of hydroxyl group(s) on the cyclohexyl ring). For example, it may be substituted at one of the oxygen atoms with an alkyl group, preferred examples being as described above. The alkyl group may be hexyl, as in 1-o-hexyl-2,3,5-trimethyl hydroquinone (HTHQ).

The benzo/hydroquinone may in particular be selected from those listed in Examples 1 and 6 below. It may for example be t-butyl hydroquinone (TBHQ) which is a para-hydroquinone substituted at the 2-position with a t-butyl group, or thymoquinone which is a para-benzoquinone substituted at the 2-position with an iso-propyl group and at the 5-position with a methyl group, or its corresponding hydroquinone, thymohydroquinone. It may be selected from TBHQ, p-hydroquinone, 2,3-dimethyl-p-hydroquinone and 2-ethyl-p-hydroquinone, more preferably from TBHQ, 2,3-dimethyl-p-hydroquinone and 2-ethyl-p-hydroquinone.

In general it is preferred that the benzo/hydroquinone is not either an unsubstituted benzoquinone or an unsubstituted hydroquinone.

A benzo/hydroquinone used in the invention, in particular thymoquinone, dithymoquinone or thymohydroquinone, is ideally used in the form of the isolated quinone (whether naturally or synthetically derived, preferably the latter) rather than as part of a plant extract containing a number of different materials.

The benzo/hydroquinone may be of the type which is active as an antioxidant.

In cases it may be preferred for the quinone to be a hydroquinone, more preferably an alkyl-substituted hydroquinone. Of these, TBHQ is particularly preferred.

A formulation according to the invention may contain more than one benzo/hydroquinone.

In one embodiment of the invention, the formulation is for use against staphylococci, in particular S. aureus. In this embodiment, the benzo/hydroquinone preferably:

a) is a para-substituted benzo/hydroquinone; and/or
b) is a hydroquinone, or at least a mixture of a hydroquinone and its corresponding benzoquinone which contains greater than 50%, more preferably greater than 60 or 70 or 80 or 90% w/w of the hydroquinone; and/or
c) has one or more, for example one or two, substituents selected from alkyl and halogen, more preferably selected from alkyl (preferred examples of such groups being as described above); and/or
d) is substituted at least at the 2-position, more preferably with a substituent selected from alkyl and halogen, yet more preferably with an alkyl group (preferred examples of such groups being as described above); and/or
e) is not substituted with more than one electron withdrawing group such as a halogen or nitro group, and is more preferably not substituted with any electron withdrawing groups; and/or
f) does not have sterically hindering substituents (in particular t-butyl groups and possibly also iso-propyl groups) positioned adjacent to both of its C—OH or C═O groups; and/or
g) is unsubstituted at least one of the two positions adjacent at least one of (preferably both of) its C—OH and C═O groups (in other words, at least one and preferably both of the C—OH or C═O groups is unsubstituted at least one of its adjacent carbon atoms); and/or
h) in particular where it is a benzoquinone, is substituted with at least one and preferably two electron donating groups, for example alkyl (in particular t-butyl or iso-propyl, preferably the former) or alkoxy (in particular methoxy or ethoxy, preferably the former) groups; and/or
i) is selected from p-benzoquinone, p-hydroquinone, TBHQ, 2-methyl-p-hydroquinone, 2,3-dimethyl-p-hydroquinone, 2-ethyl-p-hydroquinone and thymohydroquinone (2-iso-propyl-5-methyl-p-hydroquinone)- and/or
j) is selected from TBHQ, 2-methyl-p-hydroquinone, 2,3-dimethyl-p-hydroquinone, 2-ethyl-p-hydroquinone and thymohydroquinone.

The above preferences may also apply more generally, for instance when the formulation is for use against micro-organisms other than staphylococci.

In another embodiment of the invention, the formulation is for use against propionibacteria, in particular against P. acnes and more particularly for the treatment of acne, in which case the benzo/hydroquinone preferably:

a) is a para-substituted benzo/hydroquinone; and/or
b) is a hydroquinone, or at least a mixture of a hydroquinone and its corresponding benzoquinone which contains greater than 50%, more preferably greater than 60 or 70 or 80 or 90% w/w of the hydroquinone; and/or
c) has one or more, for example one or two, alkyl substituents, preferred examples being as described above; and/or
d) is substituted at least at the 2-position, more preferably with an alkyl group such as a group selected from methyl, ethyl, iso-propyl and t-butyl, yet more preferably with either methyl, ethyl or iso-propyl and most preferably with either methyl or ethyl; and/or
e) is not substituted with any electron withdrawing groups such as halogen or nitro groups; and/or
f) does not have sterically hindering substituents (in particular t-butyl groups and possibly also iso-propyl groups) positioned adjacent to both of its C—OH or C═O groups; and/or
g) is unsubstituted at least one of the two positions adjacent at least one of (preferably both of) its C—OH and C═O groups (in other words, at least one and preferably both of the C—OH or C═O groups is unsubstituted at least one of its adjacent carbon atoms); and/or
h) especially if it is a para-benzo/hydroquinone, is unsubstituted at the 5-position, or is substituted at the 5-position with a methyl group, more preferably the former; and/or
i) is selected from p-hydroquinone, p-benzoquinone, TBHQ, thymoquinone (2-iso-propyl-5-methyl-p-benzoquinone), 2-ethyl-p-hydroquinone and 2,3-dimethyl-p-hydroquinone; and/or
j) is selected from TBHQ, thymoquinone, 2-ethyl-p-hydroquinone and 2,3-dimethyl-p-hydroquinone.

The above preferences may also apply more generally, for instance when the formulation is for use against micro-organisms other than propionibacteria.

Although we do not wish to be bound by this theory, it is believed that in a formulation according to the invention, the interaction between the peroxide and the benzo/hydroquinone may involve formation of a quinone radical containing one or more C—O. groups. Such radicals, which may be at least partially responsible for the antimicrobial activity of the formulations of the invention, are likely to be resonance stabilised by substituents such as alkyl groups (in particular secondary or tertiary alkyl groups such as iso-propyl or preferably t-butyl groups) and electron donating groups. Such substituents may therefore be preferred on benzo/hydroquinones used in the invention. For a similar reason, electron withdrawing substituents may be less preferred as they may tend to destabilise quinone radicals.

To assist formation of such radicals, for instance by proton loss from a hydroquinone, it may also be preferred for at least one and preferably both of the C═O or C—OH groups of the benzo/hydroquinone to be free from steric hindrance by bulky substituents, for instance by adjacent t-butyl or iso-propyl groups.

In all the cases described above, it may be preferred for the benzo/hydroquinone not to carry any substituents in addition to those specifically referred to in each case.

In a formulation according to the invention, both the peroxide and the benzo/hydroquinone are present as active (ie, antimicrobially active) agents. It is particularly surprising that the two compounds can act together synergistically to inhibit, and often to prevent, microbial activity. Peroxides are known for use as oxidising agents whereas many quinones, in particular hydroquinones, are known for use as antioxidants. They might therefore be expected, when combined, to reduce one another's activity. Indeed in the past antioxidants have been combined with peroxides, in skin care preparations as well as in other fields, specifically for the purpose of reducing the activity of the peroxides for example as oxidising agents or catalysts.

Instead, as is shown in the examples below, these two classes of compounds have been found to increase one another's activity in a manner which can be synergistic compared to the sum of the activities of the two compounds individually. In cases where the activity of the combination is only a linear sum of the activities of the two individual compounds, nevertheless this can still have beneficial effects in that it allows the use, in for example topical skin treatment formulations, of lower levels of potentially irritant peroxides without undue loss of antimicrobial activity. This advantage would have been far from obvious in view of the apparently opposite modes of action of the two classes of compound.

It is possible that the synergy observed when a benzo/hydroquinone is combined with a peroxide may be due to the formation of a reaction product (for example, a quinone radical as described above) which has an antimicrobial activity greater than those of the individual reactants. The invention may thus embrace an antimicrobial formulation containing a reaction product formed between a benzo/hydroquinone and a peroxide selected from diacyl peroxides, alkyl hydroperoxides and metal peroxides, in particular between a benzo/hydroquinone and a diacyl peroxide such as benzoyl peroxide; this reaction product may be formed in situ immediately prior to, or at the point of, use.

In a formulation according to the invention, the peroxide and the benzo/hydroquinone, and their relative proportions, are preferably such as to yield at least an additive level of antimicrobial activity compared to the activities of the individual compounds alone (this is sometimes referred to as an “indifferent” interaction between the compounds). More preferably, the compounds and their relative proportions are such as to yield a synergistic effect on antimicrobial activity, by which is meant that the antimicrobial activity of the combination of the two compounds is greater than the sum of the individual antimicrobial activities of the same amounts of the two compounds used individually, or in some cases (depending on the test method used) that the antimicrobial activity of the combination surpasses that of the more active of its two constituents. An increased level of activity in these contexts may be manifested by a lower concentration of the active(s) being needed to inhibit and/or to kill the relevant organism, and/or by a larger zone of inhibition in a disc diffusion assay, and/or by a faster rate of microbial inhibition or killing.

Antimicrobial activity encompasses activity against micro-organisms generally, including bacteria (both Gram-positive and Gram-negative), viruses, fungi, protozoa and algae. It may be growth inhibitory activity or more preferably biocidal (ie, lethal to the relevant organism). A formulation according to the present invention is preferably active at least as a bactericide and/or fungicide (preferably at least the former), more preferably against bacteria associated with skin or skin-borne infections, yet more preferably against staphylococci (and in cases other Gram-positive cocci) and/or propionibacteria and/or suitably against other bacteria capable of causing, exacerbating or transmitting a skin or skin structure condition. Most preferably the formulation is active against strains of Staphylococcus aureus and/or Propionibacterium acnes.

In a particularly preferred embodiment of the invention, the formulation is active against bacteria associated with acne, such as P. acnes and in some instances P. granulosum. It may instead or in addition be active against Gram-positive cocci, for example staphylococci (such as those listed in the examples below, in particular S. aureus) and enterococci (such as E. faecalis and/or E. faecium, in particular the former).

In the context of this invention, activity against a particular species of micro-organism may be taken to mean activity against at least one, preferably two or more, strains of that species.

The formulation is preferably active against bacteria, in particular staphylococci and/or propionibacteria (and in some cases other Gram-positive cocci, for example enterococci), which are wholly or partially resistant to one or more antibiotics, for instance those which are in common clinical use. More particularly the formulation is preferably active against one or more erythromycin-resistant, clindamycin-resistant and/or tetracycline-resistant P. acnes strains of bacteria, and/or against one or more methicillin-resistant S. aureus (MRSA) strains, and/or against one or more vancomycin intermediate S. aureus (VISA) strains. It is preferably active at least against one or more erythromycin-resistant, clindamycin-resistant and/or tetracycline-resistant P. acnes strains,

Antimicrobial activity may be measured in conventional manner, for instance using the tests described in the examples below. Generally tests for activity involve treating a culture of the relevant micro-organism with the candidate antimicrobial compound, incubating the treated culture under conditions which would ordinarily support growth of the micro-organism, and assessing the level of growth, if any, which can occur in the presence of the candidate compound.

Preferably the peroxide used in the present invention has a minimum inhibitory concentration (MIC), at least against staphylococci and/or propionibacteria, of 500 μg/ml or less, such as from 0.5 to 500 μg/ml. Its corresponding minimum biocidal concentration (MBC) is preferably 4000 μg/ml or less, more preferably 2000 μg/ml or less, yet more preferably 1000 or 500 μg/ml or less. Suitably the ratio of its MIC to its MBC is from 0.125 to 1, ideally from 0.5 to 1.

MIC and MBC values may be measured using conventional assay techniques, for instance as described in the examples below.

Preferably the peroxide retains antimicrobial activity in the presence of at least one of, preferably two or more of, serum, lipid and salt (sodium chloride), for instance as tested in the examples below—these are species which can be present at the surface of the skin and hence performance in this context can be indicative of suitability for use in topical skin treatment formulations. Activity in the presence of lipid and sodium chloride can be especially important in the context of acne treatment; activity in the presence of serum and sodium chloride can be especially important in the context of the treatment or prevention of staphylococcal infections.

Ideally the peroxide retains at least some activity, preferably at least 50 or 60 or 70 or 80 or even 90% of its antimicrobial activity, at least against staphylococci and/or propionibacteria, in the presence of at least one of, preferably two or more of, serum, lipid and salt. Yet more preferably the antimicrobial activity of the peroxide, at least against staphylococci and/or propionibacteria, is potentiated by at least one of serum, lipid and sodium chloride. Most preferably the antimicrobial activity of the peroxide is potentiated by lipid.

Preferably the benzo/hydroquinone used in the present invention has a MIC, at least against staphylococci and/or propionibacteria, of 150 μg/ml or less, more preferably 125 or 100 μg/ml or less, yet more preferably 70 or 50 or 40 or 30 or even 20 or 10 μg/ml or less, such as from 0.5 to 100 or 50 μg/ml. Its corresponding MBC is preferably 300 μg/ml or less, more preferably 150 μg/ml or less, yet more preferably 100 or 70 or 50 or 40 or 30 or even 20 or 10 μg/ml or less. Suitably the ratio of its MIC to its MBC is from 0.125 to 1, ideally from 0.5 to 1.

Preferably the benzo/hydroquinone retains antimicrobial activity in the presence of at least one of, preferably two or more of, serum, lipid and salt (sodium chloride), for instance as tested in the examples below. Ideally it retains at least some activity, preferably at least 50 or 60 or 70 or 80 or even 90%, of its antimicrobial activity, at least against staphylococci and/or propionibacteria, in the presence of at least one of, preferably two or more of, serum, lipid and salt. Yet more preferably the antimicrobial activity of the benzo/hydroquinone, at least against staphylococci and/or propionibacteria, is potentiated by at least one of serum, lipid and sodium chloride.

Most preferably the antimicrobial activity of the benzo/hydroquinone is potentiated by lipid.

The concentration of the peroxide in a formulation according to the invention might suitably be 0.05% w/v or greater, preferably 0.1% w/v or greater. Its concentration might be up to 10% w/v, preferably below or equal to 2.5% w/v.

The concentration of the benzo/hydroquinone in the formulation might suitably be 0.05% w/v or greater, preferably 0.1% w/v or greater. Its concentration might be up to 5% w/v, preferably up to 2.5% w/v.

Due to the presence of the benzo/hydroquinone, it may be possible for the concentration of the peroxide, for example at the site of action when the formulation is applied in vivo, to be less than the SMC, or even than the MIC, of the peroxide alone. Preferably the concentration of the peroxide at this point is 0.5 or less times its MBC or MIC, more preferably 0.25 or less times. The same comments apply to the benzo/hydroquinone, which in cases may be present for example at the site of action at 0.5 or 0.25 or less times its individual MBC or MIC.

Preferably the ratio of the peroxide concentration in the formulation to that of the benzo/hydroquinone is from 1:1000 to 1000:1, more preferably from 1:10 to 100:1, yet more preferably from 1:4 to 100:1 or 10:1.

A formulation according to the invention is preferably suitable for, and more preferably adapted for, topical administration to human or animal, in particular human, skin. It may also be suitable for, or adapted for, topical administration to other epithelia such as the nares, scalp, ears, eyes, vagina and oral cavity, in particular the nares and ears. It may take the form of a lotion, cream, ointment, foam, paste or gel or any other physical form known for topical administration, including for instance a formulation which is, or may be, applied to a carrier such as a sponge, swab, brush, tissue, skin patch, dressing or dental fibre to facilitate its topical administration. It may take the form of a nasal spray or of eye or ear drops. It may be intended for pharmaceutical (which includes veterinary) use, for example to treat skin infections or as a prophylactic against infections such as MRSA, and/or for cosmetic or other non-medical care purposes (for example, for general hygiene or cleansing).

The vehicle in which the peroxide and the benzo/hydroquinone are contained may be any vehicle or mixture of vehicles which is suitable for topical application; the type chosen will depend on the intended mode and site of application. Many such vehicles are known to those skilled in the art and are readily available commercially. Examples may for instance be found in Williams' “Transdermal and Topical Drug Delivery”, Pharmaceutical Press, 2003, and other similar reference books. See also Date, A A et al, Skin Pharmacol. Physiol., 2006, 19(1): 2-16 for a review of topical drug delivery strategies.

As described above, the vehicle may be such as to target a desired site and/or time of delivery of the formulation. It may for instance target the formulation to the skin or hair follicles or to the anterior nares (the latter being particularly suitable when the formulation is used as a preventative treatment against MRSA and other staphylococcal bacteria). It may delay or otherwise control release of the formulation over a particular time period. Either or both of the peroxide and the benzo/hydroquinone may be microencapsulated, for instance in liposomes—particularly suitable liposomes, for topical use, are those made from stratum corneum lipids, eg, ceramides, fatty acids or cholesterol.

In some cases a polar vehicle may be preferred. Where the formulation is intended for use on the skin, in particular to treat skin and skin structure infections, the vehicle may be primarily non-aqueous, although in the case of an anti-acne treatment an aqueous vehicle may be used. The vehicle may be surface-active, in particular when it is intended for use in treating surfaces, for instance to cleanse instruments or working areas in particular against staphylococci. In cases the vehicle may be alcohol-based or silicon-based.

The formulation may contain standard excipients and other additives known for use in pharmaceutical or veterinary formulations, in particular topical skin care formulations. Examples include emollients, perfumes, antioxidants, preservatives and stabilisers; others may be found in Williams' “Transdermal and Topical Drug Delivery”, supra. It may further contain additional active agents such as antimicrobial agents. Where the formulation is intended for topical application to the skin, in particular to treat skin and skin structure infections and/or to treat conditions such as acne or atopic dermatitis, it may additionally contain one or more agents selected from anti-acne agents, keratolytics, comedolytics, anti-inflammatories, anti-proliferatives, antibiotics, anti-androgens, sebostatic agents, anti-pruritics, immunomodulators, agents which promote wound healing and mixtures thereof, it may instead or in addition contain one or more agents selected from sunscreens, moisturisers, emollients and mixtures thereof. Generally speaking a formulation for use according to the invention may contain one or more agents which enhance the activity of another active agent present in the formulation, or reduce a side effect of such an active, or improve patient compliance on administration of the formulation.

An additional antimicrobial agent may for example be selected from the group consisting of biocides, disinfectants, antiseptics, antibiotics, antimicrobially active antioxidants and mixtures thereof; it is preferably active as a bactericide, in particular against propionibacteria and/or staphylococci. It may be active as an anti-mycotic.

It may however be preferred for the peroxide(s) and the benzo/hydroquinone(s) to be the only active agents in the formulation, or at least to be the only antimicrobially or antibacterially active agents.

A formulation according to the invention may be suitable for, more preferably adapted for, use on a surface other than living tissue, for instance to treat floors or walls (whether internal or external), work surfaces or instruments, to disinfect contact lenses or to cleanse hair or teeth or nails so as to reduce microbe levels. It may be suitable for application to growing or harvested crops, foodstuffs, non-living tissue (for instance for use as a preservative), bedding or clothing (for instance for bio-agent decontamination). In these cases the excipients, vehicles and/or other additives included with the peroxide and the benzo/hydroquinone may be different to those included in a topical skin care formulation, but again may be conventional as known for use in such contexts.

The formulation may be incorporated into, and hence applied in the form of, another product such as a cosmetic, a skin or hair care preparation, a pharmaceutical (which includes veterinary) preparation, a toiletry product (for instance a bath or shower additive or a cleansing preparation), a laundry or other fabric treatment product or an agricultural or horticultural product.

The formulation may be suitable for incorporation into another product as a preservative; it may for example be included in a food or beverage, a pharmaceutical preparation, a cosmetic or toiletry product, or a tissue, serum or other body sample, so as to inhibit or prevent microbial activity in the product.

The invention provides, according to a second aspect, a product which incorporates an antimicrobial formulation according to the first aspect.

In some cases it may be preferred for a formulation according to the invention not to contain any cross-linkable thermoplastic or elastomeric polymers such as those disclosed in U.S. Pat. No. 6,069,208, and/or not to contain any sulphur accelerators as disclosed in that document.

In some cases it may be preferred for a formulation according to the invention not to contain polymers of the type disclosed in GB-1 089 428, in particular those prepared by reacting an organotin compound with a glycol monoacrylate monoester of a dicarboxylic acid.

In some cases it may be preferred for a formulation according to the invention not to contain both benzoyl peroxide and a benzo/hydroquinone selected from t-butyl catechol, hydroquinone, toluhydroquinone, p-benzoquinone, TBHQ, 2,5-di-t-butyl-hydroquinone and hydroquinone monomethyl ether.

In some cases, it may be preferred for a formulation according to the invention not to contain plasticisers and/or surfactants and/or calcium carbonate, in particular for the formulation not to contain plasticisers. It may be preferred for the formulation not to be a benzoyl peroxide-containing formulation of the type specifically disclosed in WO-97/32845.

A formulation according to the invention may be prepared in situ, at or immediately before the point of its application for instance to the skin or another surface. Thus according to a third aspect, the present invention provides a kit for preparing an antimicrobial formulation, preferably a formulation according to the first aspect, the kit comprising (a) a source of a peroxide selected from diacyl peroxides, alkyl hydroperoxides and metal peroxides and (b) a source of a benzoquinone or hydroquinone, together with instructions for combining the two compounds so as to make the formulation at or before the point of intended application, and/or for the co-administration of the two compounds to a surface such as the skin. The peroxide and the benzo/hydroquinone may each be present in a suitable respective vehicle.

According to one embodiment, the formulation or kit of the invention may contain both a peroxide of the defined type and a benzo/hydroquinone, each encapsulated (for instance microencapsulated) in a separate delivery vehicle; this might for instance allow their release, and hence their contact with one another, only at the intended site of administration.

A fourth aspect of the invention provides a method for preparing an antimicrobial formulation, which method involves mixing together (a) a peroxide selected from diacyl peroxides, alkyl hydroperoxides and metal peroxides and (b) a benzoquinone or hydroquinone, preferably together with a pharmaceutically acceptable vehicle as described above.

According to a fifth aspect of the invention there is provided a formulation (preferably a formulation according to the first aspect of the invention) containing (a) a peroxide selected from diacyl peroxides, alkyl hydroperoxides and metal peroxides and (b) a benzoquinone or hydroquinone, for use in the treatment (preferably the topical treatment) of a condition which is either caused, exacerbated or transmitted by microbial activity, in particular bacterial or fungal activity, more particularly bacterial activity, most particularly staphylococcal and/or propionibacterial activity. The condition is preferably a skin or skin structure condition, for example acne.

In the context of the present invention, treatment of a condition encompasses both therapeutic and prophylactic treatment, of either an infectious or a non-infectious condition, in either a human or an animal and in particular on the skin. It thus involves use of the formulation as a microbicide, preferably as a bactericide, most preferably against staphylococci and/or propionibacteria and/or enterococci.

Skin and skin structure conditions which might be treated according to the invention include acne, infected atopic eczema, superficial infected traumatic lesions, wounds, burns, ulcers, folliculitis, mycoses and other superficial primary and secondary skin and skin structure infections. In particular the formulation may be for use in treating acne or acne lesions (for instance, to reduce acne-related scarring).

Treatment of acne encompasses the treatment and/or prevention of lesions and/or scarring associated with acne. Acne is a multifactoral disease of the pilosebaceous follicles of the face and upper trunk, characterised by a variety of inflamed and non-inflamed lesions such as papules, pustules, nodules and open and closed comedones. Its treatment can therefore encompass the treatment of any of these symptoms.

In general, a formulation according to the invention will be used for the treatment of symptoms which are directly due to acne rather than for instance infections which may arise as a consequence of treating acne with other actives such as antibiotics.

In the context of the present invention, “skin or skin structure condition” may in some cases encompass a condition affecting other epithelia such as in the nares, scalp, vagina, eyes, ears or oral cavity. In most cases, however, a skin or skin structure condition will be one affecting the skin or skin structure directly.

A formulation according to the invention may also be used as a therapeutic or prophylactic treatment for any area of the body—in particular the skin or nares—against staphylococci, which might otherwise cause for example MRSA-associated infections, or infections in pre-existing lesions such as eczematous lesions.

Other examples of conditions which may be treated in accordance with the fifth aspect of the invention include oral, ocular, aural, nasal and vaginal conditions. Again, treatment of such conditions encompasses both therapeutic and prophylactic treatment, of either an infectious or a non-infectious condition, in either a human or an animal but in particular a human. In particular it encompasses the prophylactic treatment of any area of the body, in particular the skin or nares, against microbial and especially bacterial infections.

Treatment of a condition may involve complete or partial eradication of the condition, removal or amelioration of associated symptoms, arresting subsequent development of the condition, and/or prevention of, or reduction of risk of, subsequent occurrence of the condition.

According to the fifth aspect of the invention, the formulation of peroxide and benzo/hydroquinone may be prepared in situ, at or immediately before the point of administration. This aspect of the invention thus pertains to any use, together, of a peroxide of the defined type and a benzo/hydroquinone in the treatment (preferably the topical treatment) of a condition which is either caused, exacerbated or transmitted by microbial activity, the two compounds being administered either simultaneously or sequentially. Again the condition is preferably a skin or skin structure condition, and/or a condition for which the associated microbes are present on the skin.

According to a sixth aspect, the invention provides the use together of (a) a peroxide selected from diacyl peroxides, alkyl hydroperoxides and metal peroxides and (b) a benzoquinone or hydroquinone, in the manufacture of a medicament for the treatment of a condition which is either caused, exacerbated or transmitted by microbial (especially bacterial or fungal, more especially bacterial) activity, in particular staphylococcal and/or propionibacterial activity. The condition is typically a skin or skin structure condition, more typically acne. It may be a staphylococcal infection, in particular a skin-borne infection. The medicament is suitably for topical use.

The invention further provides, according to a seventh aspect, the use together of a peroxide of the defined type and a benzo/hydroquinone, as an antimicrobial agent, in particular as a bactericide or fungicide, or in the manufacture of an antimicrobial or specifically bactericidal/fungicidal formulation.

An eighth aspect provides a method for controlling the growth of a micro-organism, in particular a bacterial or fungal micro-organism, more particularly a bacterial micro-organism and most particularly a staphylococcal bacterium or Propionibacterium, the method comprising applying, to an area infected or suspected to be infected or capable of becoming infected with the micro-organism, (a) a peroxide selected from diacyl peroxides, alkyl hydroperoxides and metal peroxides and (b) a benzoquinone or hydroquinone. Again the two compounds may be applied simultaneously or sequentially.

In this context, “controlling the growth” of a micro-organism embraces inhibiting or preventing its growth, whether completely or partially, as well as killing either completely or partially a culture of the organism. It also embraces reducing the risk of subsequent growth of the organism in the area being treated. The method of the invention may thus be used to treat an existing occurrence of the organism or to prevent a potential subsequent occurrence.

The method of the eighth aspect of the invention is preferably not for controlling the growth of an aquatic micro-organism, in particular an aquatic organism of the type referred to in US-2003/0012804.

Again the area to which the peroxide and the benzo/hydroquinone are applied will typically be a surface such as human or animal tissue, in particular the skin or nares, typically of a living human or animal. In this case the two compounds may be applied for therapeutic purposes or for non-therapeutic (eg, purely cosmetic) purposes. Alternatively it may be a non-living surface such as in a hospital or food preparation area. For example the method of the eighth aspect of the invention may be used to treat work surfaces, surgical or other instruments, surgical implants or prostheses, contact lenses, foods, crops, industrial plant, floors and walls (both internal and external), bedding, furniture, clothing and many other surfaces.

The method of the eighth aspect of the invention embraces a method for controlling microbial growth in or on a human or animal patient, the microbes typically being staphylococci and/or propionibacteria and the growth typically being controlled on the skin or in cases on other epithelia such as the nares.

The method of the eighth aspect preferably involves applying a formulation according to the first aspect of the invention.

According to a ninth aspect, the invention provides a method for controlling the growth of a micro-organism, in particular a bacterial or fungal organism, most particularly a bacterial organism, in a product which contains or is suspected to contain or is capable of containing the micro-organism, the method comprising incorporating into the product a combination of (a) a peroxide selected from diacyl peroxides, alkyl hydroperoxides and metal peroxides and (b) a benzoquinone or hydroquinone. The product may for example be a food or beverage, a pharmaceutical (which includes veterinary) preparation, a cosmetic or toiletry product, or an agricultural or horticultural product.

Thus the method of the ninth aspect of the invention may be used to preserve all manner of products, or it may be used in the sanitation of food or water supplies or the disinfection of farming areas.

A tenth aspect of the invention provides the use of a benzoquinone or a hydroquinone in an antimicrobial formulation, in combination with a peroxide of the above defined type, for the purpose of increasing the antimicrobial (in particular antibacterial and/or antifungal, more particularly against staphylococci and/or propionibacteria) activity of the formulation and/or of reducing the amount of peroxide in the formulation without undue loss of antimicrobial activity.

An increase in antimicrobial activity may be as compared to that of the peroxide alone, at the same concentration as used when combined with the benzo/hydroquinone. Ideally the increase is as compared to the sum of the activities of the peroxide and the benzo/hydroquinone individually, again at the same respective concentrations as used when the two are combined.

A reduction in the amount of peroxide in the formulation may be as compared to the amount which would otherwise have been used in the formulation in order to achieve a desired level of activity, in particular in order to have acceptable efficacy in the context of its intended use. The reduction may be manifested by reduced side effects which would otherwise have been observed during use of the formulation, in particular reduced skin irritancy. According to the invention, the benzo/hydroquinone may therefore be used for the dual purposes of reducing the skin irritancy or other undesired properties of the formulation, without or without undue loss of antimicrobial activity.

Preferably the benzo/hydroquinone is used without any reduction in antimicrobial activity compared to the level exhibited by the formulation prior to addition of the quinone. More preferably it is used to give an increase in antimicrobial activity. It may however be used to reduce the amount of peroxide present, and/or its associated side effects, whilst maintaining the antimicrobial activity of the resultant formulation at a level, albeit lower than that which it would otherwise have exhibited, which is still acceptable in the context of its intended use.

An eleventh aspect of the invention provides the use of a peroxide of the above defined type in an antimicrobial formulation, in combination with a benzoquinone or a hydroquinone, for the purpose of increasing the antimicrobial activity of the formulation and/or of reducing the amount of benzo/hydroquinone present in the formulation without or without undue loss of antimicrobial activity. Similar comments apply to this aspect of the invention as to the tenth aspect, but vice versa.

Preferred features of the second and subsequent aspects of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Moreover unless stated otherwise, any feature disclosed herein may be replaced by an alternative feature serving the same or a similar purpose.

The present invention will now be described by way of example only and with reference to the accompanying illustrative drawings.

### BRIEF DESCRIPTION OF THE DRAWINGS

FIG. 1 is an isobologram showing FIC (fractional inhibitory concentration) values for mixtures of benzoyl peroxide (BP) and t-butyl hydroquinone (TBHQ) against a staphylococcal bacterial strain, as measured in Example 2 below, and

FIG. 2 is an isobologram showing FIC values for mixtures of BP and TBHQ against a propionibacterial strain, as measured in Example 7 below.

### DETAILED DESCRIPTION

Experimental tests were conducted to determine the antimicrobial activities of formulations according to the invention. As a comparison, the antimicrobial activities of formulations containing a benzo/hydroquinone or a peroxide alone were also measured.

Test Micro-Organisms

The following test micro-organisms were used, representing a spectrum of organisms against which formulations of the present invention may be used.

1. Staphylococcus aureus—the principal staphylococcal test micro-organism used in these studies was Staphylococcus aureus ATCC 29213. This strain is the one recommended for QC/QA purposes in Minimum Inhibitory Concentration (MIC) assays by the US Clinical and Laboratory Standards Institute (formerly the NCCLS), an FDA-recognised body. S. aureus ATCC 29213 is susceptible to beta-lactam antibiotics such as methicillin and to many other antibiotics in clinical use worldwide today.

Other staphylococcal strains were also tested, as described in Example 5 below. These included certain antibiotic resistant staphylococci, such as the methicillin resistant S. aureus (MRSA) strains EMRSA-15 and EMRSA-16, both available from the Central Public Health Laboratory (CPHL), Colindale, UK. These strains are resistant not only to all beta-lactams but also to a number of other antibiotics in clinical use, making them a serious threat to human health. They are also responsible for the majority (>95%) of hospital-acquired MRSA infections in the UK.

S. aureus and other staphylococci are common causes of a wide range of skin, skin structure and wound infections. S. aureus itself is also known to exacerbate eczema.

2. Propionibacterium spp.—the principal propionibacterial strain used in these studies was Propionibacterium acnes NCTC 737. This is the type strain of the genus; it is fully susceptible to antibiotics.

The propionibacteria are clinically significant due to their involvement in acne. This is a very common, complex and multi-factorial skin disease in which P. acnes and other Propionibacterium spp. (for example P. granulosum) play key roles. They are also opportunistic pathogens in compromised hosts.

For the FIC (fractional inhibitory concentration—see explanation below) assays of Example 7, NCTC 737 was replaced by its close relative P. granulosum (an in-house strain referred to as PRP-055).

Other propionibacterial strains were also tested, as described in Example 8 below. These included certain antibiotic resistant propionibacteria, such as the two P. acnes strains PRP-010 and PRP-053 which are resistant respectively to macrolides-lincosamides-streptogramins-ketolides (MLSK) and to macrolides-lincosamides-streptogramins (MLS) and tetracycline—in other words, PRP-010 is resistant to erythromycin and clindamycin, and PRP-039 to erythromycin, clindamycin and tetracycline.

In addition, certain strains of P. granulosum and a type strain of P. avidum, both other propionibacteria involved in acne, were also tested in Example 8.

3. Enterococcus faecalis ATCC 29212—this is a Gram-positive bacterium belonging to the genus Enterococcus. Enterococci have similar properties to streptococci, but differ in their ability to grow on bile-salt containing media such as MacConkey's Agar. Their principal habitat is the mammalian gastrointestinal tract. They cause a number of important infections including endocarditis, urinary tract infections and abscesses. In the context of skin, they are frequently isolated from wound infections. Unlike the streptococci, the enterococci have developed widespread resistance to penicillin. More recently, E. faecalis and E. faecium strains have also developed resistance to the glycopeptide antibiotics such as vancomycin. Vancomycin-Resistant Enterococci (VRE), principally vanA strains of E. faecium, now represent a serious healthcare-acquired infection hazard in the USA, Japan and Western Europe.

Activity observed against the above micro-organisms is expected to be a reasonable qualitative predictor of antimicrobial activity, in particular against micro-organisms responsible for skin and skin structure infections.

The staphylococci and enterococci were cultured and maintained on Mueller-Hinton Medium (agar and broth) at pH 7.2; they were incubated aerobically at 37° C. for 24 hours.

The Propionibacterium spp organisms were cultured and maintained on Wilkins-Chalgren Anaerobe Medium (agar and broth) at pH 6.0; cultures were incubated anaerobically at 37° C. for 72 hours.

The following tests were carried out to assess antimicrobial activity against these organisms.

(a) Minimum Inhibitory Concentration (MIC) Assay

This is a standard international method for quantitatively assessing the antimicrobial activity of a compound in a liquid medium. The method used a 96-well microtitre plate, capable of holding about 200 μl of liquid per well. The wells contained liquid culture medium and ranges of decreasing concentrations of the relevant test compound in doubling dilutions (eg, 1000, 500, 250, 125 . . . μg/ml, etc. down to 1.95 μg/ml). The culture media were as described above for the relevant test organisms.

The wells were inoculated with a liquid suspension of freshly grown micro-organism and incubated under the conditions described above. After incubation, the microtitre plate was examined visually (with the aid of a light box) for cell pellets in and/or opaque wells, which would indicate microbial growth. The MIC value was recorded as the lowest concentration of test compound required to inhibit microbial growth, ie, the lowest concentration for which the liquid in the well remained clear.

The assays were conducted in duplicate and included both negative (culture medium only) and positive (culture medium, diluting solvent and inoculum) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism

(b) Minimum Bactericidal Concentration (MBC) Assay

This assay, normally carried out after an MIC assay, determines the minimum concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on non-selective agar medium, under the incubation conditions described above. Following incubation they were examined visually for bacterial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or be overcome by natural (ie, innate) antimicrobial resistance.

(c) Disc Diffusion Assay (DDA)

This is an internationally recognised standard method for qualitatively assessing the antimicrobial activity of a compound.

A sterile paper disc was impregnated with a sample of the test compound and a minimum of 30 minutes allowed for the solvents to evaporate as far as possible. The disc was then placed on an agar plate onto which the test micro-organism had been inoculated. The plate was then incubated under the conditions described above, following which it was examined visually for signs of bacterial growth. If the test compound had antimicrobial activity, a circular zone of no growth would be obtained around the disc. The diameter of this zone of “inhibition” was measured using a ProtoCOL™ automated zone sizer (Synbiosis, Cambridge, UK). In general, a greater diameter and/or area of the zone of inhibition indicates a greater antimicrobial activity in the relevant test compound, although other factors such as test compound mobility through the agar gel may also influence the result.

The area of the zone of inhibition was calculated from the measured zone diameter (D) using the formula π(D/2)².

(d) Synergy Disc Diffusion Assay (SDDA)

This is a variation on the DDA method, in which two compounds are tested together for their combined antimicrobial activity. It provides a qualitative indication as to whether the compounds are likely to interact synergistically.

Two test compounds A and B were placed on a single paper disc and the above described DDA procedure repeated. An increase in diameter of the zone of inhibition, compared to the greater of the zone diameters for the two compounds individually, was taken to indicate potential antimicrobial synergy. In practical terms, an increase of greater than 5 mm was treated as significant. The larger the increase in zone size, the greater the likelihood of a synergistic interaction between the two test compounds.

(e) Supplemented Disc Diffusion Assays

Either the DDA or the SDDA tests may be carried out using an agar gel supplemented with blood, lipid and/or salt to simulate some of the major components present in human skin and to assess whether these substances might reduce the antimicrobial activity observed for the test compounds. Performance under these conditions can provide a more reliable indication of activity on topical application. For assays conducted using S. aureus strains, the supplements could for example be defibrinated horse blood (5% v/v) and sodium chloride (100 mM). For those using Propionibacterium spp. strains, the supplements could be lipid (Tween™ 80 at 1% v/v) and sodium chloride (100 mM).

(f) Fractional Inhibitory Concentration (FIC) Assay

This assay was used to determine the mode of interaction between two antimicrobial compounds A and B. It was similar to the MIC assay, utilising a 96-well microtitre plate and liquid culture medium. The test compounds were added together to each well at a range of concentrations starting at their respective MIC values and descending in doubling dilutions as with the MIC assay. Typically an 8×8 array of wells could be used to combine 8 different concentrations of compound A (from its MIC downwards, including zero) with 8 different concentrations of compound B (ditto).

The wells were inoculated with freshly grown micro-organism and incubated under the conditions described above.

As for the MIC assay, the results were read by the naked eye. A minimum inhibitory concentration was recorded for each combination of A and B. A fractional FTC index (FICI) was then calculated for each compound in that mixture, and these two indices were added together to give an overall FICI indicative of the mode of interaction.

Thus for each mixture tested, the FTC for compound A (FIC_{A})=MIC for (A+B)/MIC for A alone. Similarly the FTC for compound B (FIC_{B})=MIC for (A+B)/MIC for B alone. The overall FICI=FIC_{A}+FIC_{B}.

An FICI of 0.5 or less was taken to indicate synergy, a value from 0.5 to 4.0 an indifferent effect and values greater than 4.0 antagonism (ie, the two compounds counter one another's activity, leading overall to a diminished antimicrobial effect) (see Odds F C, “Synergy, antagonism, and what the chequerboard puts between them”, J Antimicrob. Chemother., 2003; 52:1). These results can be depicted visually on a plot (isobologram) of FIC_{A} against FIC_{B} for the mixtures tested.

(g) Time-to-Kill (TTK) Assay

This quantitative assay was designed to assess the time taken for a test compound to kill a test micro-organism.

Samples from an incubated liquid culture containing the relevant test compound and micro-organism were taken at timed intervals and inoculated onto agar plates. The plates were then incubated as described above and subsequently examined visually for growth. The numbers of viable microbial colonies on the plates were counted and converted to colony-forming units per ml (cfu/ml) using the appropriate dilution factor. By way of example, a colony count of 25 colonies from an agar plate carrying 100 μl of inoculum which had been serially diluted to 10⁻⁶ would yield a viable cell count of 25×10 (to correct to 1 ml)×106 which would be equivalent to 2.5×10⁸ cfu/ml. These cfu values were then converted into log₁₀ values and plotted graphically against time of sample removal.

At each time point, samples were assessed in triplicate; the final cfu/ml value was an average (mean) of the three readings.

An appropriate concentration of test compound(s) for use in this assay was determined based on previously conducted MIC/FIC assays.

The TTK assay can provide another measure of synergy, as combinations of compounds may interact to kill test micro-organisms more quickly than the individual compounds alone. This would be indicated by a steeper decline in viable bacterial cell counts as compared to those observed for the individual compounds alone.

(h) Matrix Time-to-Kill (MTTK) Assay

This assay can provide a further indication of antimicrobial synergy between two test compounds X and Y, revealing combinations of the two which are able to kill a micro-organism more efficiently than could either of the compounds individually when used at the same concentration.

Each of the two test compounds was dissolved, in an appropriate solvent, to 80× the initial concentration required. The concentration range for each compound was from 2× its MBC to 0.125× its MBC, with the intervening concentrations obtained by a series of doubling dilutions.

A 96-well microtitre plate was used to create a 6×6 array of samples, combining different concentrations of the two test compounds. A suitable layout for the test wells is shown below—here concentrations of the test compound X (MBC 125 μg/ml) are indicated in normal type, and concentrations of compound Y (MBC 16 μg/ml) in bold type.

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | |
|---|---|---|---|---|---|---|---|---|
| A | 0 | 15.6 | 31.25 | 62.5 | 125 | 250 | NOT IN USE | Media Only |
| | 0 | 0 | 0 | 0 | 0 | 0 | | (- |
| | | | | | | | | control) |
| B | 0 | 15.6 | 31.25 | 62.5 | 125 | 250 | | Media Only |
| | 2.0 | 2.0 | 2.0 | 2.0 | 2.0 | 2.0 | | (- |
| | | | | | | | | control) |
| C | 0 | 15.6 | 31.25 | 62.5 | 125 | 250 | | Media Only |
| | 4.0 | 4.0 | 4.0 | 4.0 | 4.0 | 4.0 | | (- |
| | | | | | | | | control) |
| D | 0 | 15.6 | 31.25 | 62.5 | 125 | 250 | | Media Only |
| | 8.0 | 8.0 | 8.0 | 8.0 | 8.0 | 8.0 | | (- |
| | | | | | | | | control) |
| E | 0 | 15.6 | 31.25 | 62.5 | 125 | 250 | | Media Only |
| | 16.0 | 16.0 | 16.0 | 16.0 | 16.0 | 16.0 | | (- |
| | | | | | | | | control) |
| F | 0 | 15.6 | 31.25 | 62.5 | 125 | 250 | | Media Only |
| | 32.0 | 32.0 | 32.0 | 32.0 | 32.0 | 32.0 | | (- |
| | | | | | | | | control) |

| | | |
|---|---|---|
| G | NOT IN USE | Media |
| | | Only (- |
| | | control) |
| H | | Media |
| | | Only (- |
| | | control) |

The test compounds, at their relevant dilutions, were added to the appropriate wells in 5 μl doses. To those wells in which a test compound should be at 0 concentration, 5 μl of solvent alone were added. Each well was also supplied with 190 μl of a suitable broth (dependent on the test micro-organism—Mueller-Hinton broth for S. aureus, for example), the contents thoroughly mixed and 100 μl volumes discarded.

To each well was then added 100 μl of an inoculum containing the relevant test micro-organism, in the same broth. The inoculum was diluted to give ca. 1×10⁷ cfu/ml.

All 36 samples were incubated under conditions appropriate for the test micro-organism, as described above. 10 μl samples were removed from each test well at time t=0, t=5 hours and t=24 hours. Each of these samples was added to 90 μl of an appropriate broth, and a series of seven further 10-fold dilutions carried out so as to yield eight samples with dilutions from 10⁻¹ to 10⁻⁸. From each of these, three 10 μl sub-samples were then plated onto individual agar plates, which were incubated under appropriate conditions (again as described above).

Following incubation, colonies were counted at an appropriate serial dilution (5-50 individual colonies visible) with the aid of a colony counter (Stuart™ Colony Counter SC6, Barloworld Scientific Ltd, Stone, UK). These measurements were then converted to numbers of colony forming units (cfu), using the formula: cfu/ml=number of colonies×serial dilution factor×100 (as only a 10 μl sample was taken). For each test combination at each time point, the final cfu/ml value was the mean of the three replicates.

For the t=24 hours sampling timepoint, the results of these assays were plotted as a matrix showing how microbial growth was affected by each combination of the test compounds X and Y. The compounds were taken to exhibit antimicrobial synergy in cases where, after 24 hours, there was a ≧2 Log₁₀ reduction in cfu/ml between the combination (X+Y) and its most active constituent, so long as the number of surviving organisms in the presence of the combination was ≧2 Log₁₀ cfu/ml below the number in the starting inoculum.

### EXAMPLE 1

### Activity Against S. aureus—MIC, MBC & (S)DDA Assays

The following experiments all used S. aureus ATCC 29213 as the test organism.

MIC, MBC and DDA assays, as described above, were carried out using the test compound benzoyl peroxide (BP) and a range of different benzoquinones and hydroquinones. Supplemented DDA assays, in the presence of salt, lipid and blood, were also conducted.

Each of the quinones was then subjected, in combination with BP, to the SDDA assay described above. In each case, increases in zone diameter (mm) and area (%) were measured with respect to those observed for the compound showing the larger zone diameters during the previous disc diffusion assays on the individual compounds.

For most (S)DDA assays, 200 μg of each compound was loaded onto each disc. The exceptions were the thymoquinone assays, in which only 50 μg of the benzoquinone was used. The solvents used were DMSO (for benzoyl peroxide, 2-methyl-p-hydroquinone, 2,3-dimethyl-p-hydroquinone and 2-ethyl-p-hydroquinone) and ethanol (for TBHQ, thymoquinone, p-hydroquinone, p-benzoquinone and thymohydroquinone).

The MIC, MBC and DDA results are shown in Table 1 below and the SDDA results in Table 2. All results are collated from a number of experiments.

TABLE 1

| | | | | DDA + | DDA + | DDA + |
|---|---|---|---|---|---|---|
| | MIC | MBC | DDA | salt | lipid | blood |
| Test compound | (μg/ml) | (μg/ml) | (mm) | (mm) | (mm) | (mm) |
| benzoyl peroxide | 250 | 250 | 11.03 | n/a | n/a | n/a |
| | | | (±0.19) | | | |
| TBHQ | 7.8 | 7.8 | 41.77 | 54.16 | 31.44 | 10.89 |
| | | | (±2.01) | | | |
| thymoquinone | 7.8 | 15.6 | 15.64 | 20.54 | 18.99 | 0.0 |
| | | | (±1.03) | | | |
| p-hydroquinone* | 62.5 | 62.5 | 18.94 | 16.61 | 15.56 | 14.01 |
| | | | (±0.84) | | | |
| p-benzoquinone | 31.25 | 31.25 | 29.34 | 27.49 | 29.05 | 20.31 |
| | | | (±0.19) | | | |
| 2-methyl-p-hydroquinone | 15.6 | 15.6 | 25.70 | 22.49 | 24.68 | 18.74 |
| | | | (±0.64) | | | |
| 2,3-dimethyl-p-hydroquinone | 7.8 | 7.8 | 33.41 | 34.24 | 35.80 | 14.32 |
| | | | (±0.48) | | | |
| 2-ethyl-p-hydroquinone | 7.8 | 15.6 | 21.06 | 24.59 | 23.04 | 17.74 |
| | | | (±0.48) | | | |
| thymohydroquinone | 7.8 | 15.6 | 57.62 | 57.59 | 41.71 | 10.27 |
| | | | (±2.67) | | | |

*Data variable: synergy observed in some tests and not in others

TABLE 2

| | SDDA | SDDA | |
|---|---|---|---|
| | with BP | increase | SDDA area |
| Test quinone | (mm) | (mm) | increase (%) |
| TBHQ | 68.54 | 26.77 | 169.30 |
| | (±2.58) | | |
| thymoquinone | 23.24 | 7.60 | 120.91 |
| | (±0.81) | | |
| p-hydroquinone | 33.36 | 14.42 | 210.15 |
| | (±0.48) | | |
| p-benzoquinone | 34.38 | 5.03 | 37.25 |
| | (±0.56) | | |
| 2-methyl-p-hydroquinone | 32.77 | 7.07 | 62.56 |
| | (±0.32) | | |
| 2,3-dimethyl-p-hydroquinone | 41.25 | 7.83 | 52.39 |
| | (±0.66) | | |
| 2-ethyl-p-hydroquinone | 30.19 | 9.13 | 105.49 |
| | (±0) | | |
| thymohydroquinone | 75.18 | 17.56 | 70.26 |
| | (±3.58) | | |

The data in Tables 1 and 2 show that each of the benzo/hydroquinones alone is active against S. aureus ATCC 29213, some strongly so—in particular the substituted benzo/hydroquinones appear to be more active than their unsubstituted counterparts, as indicated by the MIC/MBC results. Activity is maintained, at least to some extent, in the presence of salt, lipid and serum. BP alone is much less, if at all, active against the organism.

When BP is combined with a benzo/hydroquinone however, the SDDA data indicate a potential synergistic antimicrobial interaction between the two, in each case with a significant increase in zone diameter over that exhibited by either compound alone.

The BP/TBHQ SDDA assay was repeated in the presence of salt and blood, as described above. Antibacterial synergy appeared to be retained under these supplemented conditions, the zone diameter increase being 12.34 mm and the area increase 186.0%.

Thus in the presence of a suitable benzo/hydroquinone, the otherwise relatively inactive peroxide can be made very active against S. aureus. Moreover this synergy is also likely to be retained on topical application to the skin.

### EXAMPLE 2

### Activity Against S. aureus—FIC Assays

Mixtures of BP and the alkyl-substituted hydroquinone TBHQ, containing various relative proportions of the two actives, were then subjected to FIC assays against S. aureus ATCC 29213, as described above, and the results used to prepare FIC isobolograms. Acetone was used as the solvent for BP and ethanol for TBHQ.

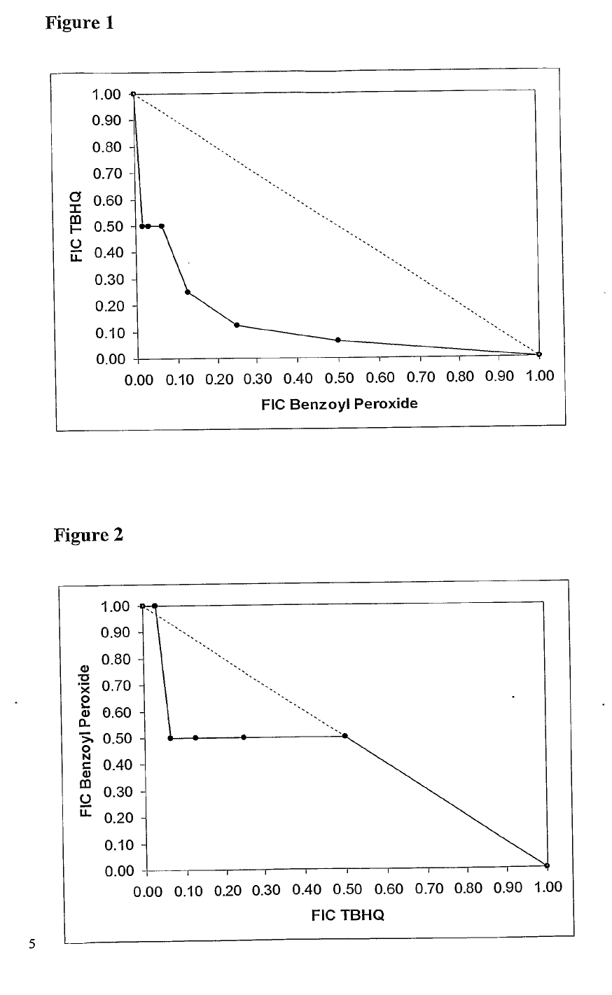

The lowest FICIs obtained for the mixtures ranged from 0.38 to 0.5, again indicating a synergistic interaction. A representative isobologram is shown in FIG. 1; the dashed line indicates where overall FICIs (ie, FIC_{BP}+FIC_{TBHQ}) equal 1, which would indicate a purely additive effect. FIG. 1 clearly demonstrates the synergistic activity of the combination of BP and TBHQ.

### EXAMPLE 3

### Activity Against S. aureus—TTK Assays

TTK assays were then conducted, as described above, on samples of BP, TBHQ and a BP/TBHQ mixture, using S. aureus ATCC 29213 as the test organism. The solvents used were DMSO for the BP and ethanol for the TBHQ. The results are shown in Table 3. cfu values were measured at time 0, 0.5 hours and 1 hour.

TABLE 3

| BP | TBHQ | CFU/ml | | | |
|---|---|---|---|---|---|
| (μg/ml) | (μg/ml) | t = 0 | 0.5 | 1.0 | Time to kill (h) |
| 0 | 0 | 7.63 × 10⁵ | 8.83 × 10⁵ | 9.27 × 10⁵ | n/a |
| 125 | 0 | 7.63 × 10⁵ | 6.20 × 10⁵ | 3.67 × 10⁴ | >1 |
| 0 | 15.6 | 7.63 × 10⁵ | 7.40 × 10⁵ | 8.47 × 10⁵ | >1 |
| 125 | 15.6 | 7.63 × 10⁵ | 1.67 × 10⁵ | 0 | 1.0 |

These data show that a combination of peroxide and hydroquinone can kill the S. aureus bacteria more quickly than either compound alone when used at the same concentration as in the mixture. This provides further evidence of antimicrobial synergy between the two agents.

### EXAMPLE 4

### Activity Against S. aureus—MTTK Assays

Samples containing BP, TBHQ and BP/TBHQ mixtures were also subjected to MTTK assays as described above, using S. aureus ATCC 29213 as the test organism. The BP was dissolved in DMSO and the TBHQ in ethanol.

The results, after 24 hours, are shown in Table 4 below. (The initial starting inoculum contained 3.25×10⁷ cfu/ml; 1×10³ cfu/ml represented the lower detection limit.)

TABLE 4
Benzoyl Peroxide

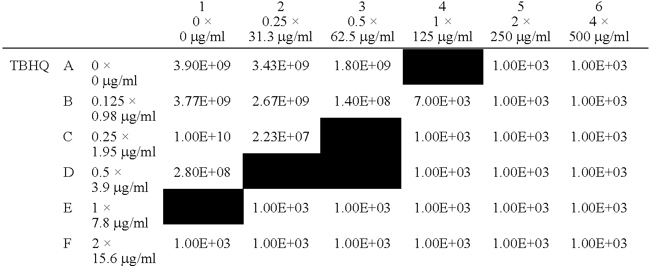

Table 4 identifies three synergistic mixtures of BP and TBHQ, as highlighted in bold on a dark grey background. The light grey cells indicate the lowest concentration of each individual active which is sufficient to kill on its own (ie, the MBC). For the synergistic mixtures, the reduction in microbial activity was greater (after 24 hours) than that obtained using either BP or TBHQ alone at the same concentrations as in the mixture.

The three synergistic BP/TBHQ combinations were those containing:

a) 0.25×MBC of BP (31.25 μg/ml)+0.5×MBC of TBHQ (3.9 μg/ml)
b) 0.5×MBC of BP (62.5 μg/ml)+0.25×MBC of TBHQ (1.95 μg/ml)
c) 0.5×MBC of BP (62.5 μg/ml)+0.5×MBC of TBHQ (3.9 μg/ml).

This further confirms that the peroxide and the hydroquinone may be used together, each at a concentration lower than its individual MBC, to counter staphylococci.

This further confirms that the peroxide and the hydroquinone may be used together, each at a concentration lower than its individual MBC, to counter staphylococci. It is likely that mixtures containing higher concentrations of either test compound will also act synergistically against S. aureus ATCC 29213.

### EXAMPLE 5

### Activity Against Other Staphylococci—MIC, MBC & (S)DDA Assays

The activities of BP, TBHQ and combinations of the two were tested against other staphylococcal bacterial strains, including some with known antibiotic resistance. MIC, MBC and (S)DDA assays were carried out as described above for each of the strains.

For all (S)DDA assays, 200 μg of each compound was loaded onto each disc. The solvents used were DMSO for the BP and ethanol for the TBHQ.

The MIC and MBC results are shown in Table 5 below and the (S)DDA results in Table 6. All results are collated from a number of experiments. Table 5 indicates the resistance phenotype for each of the test strains, some of which are resistant to many commonly used antibiotics.

TABLE 5

| | | | BP | TBHQ | TBHQ |
|---|---|---|---|---|---|
| | | BP MIC | MBC | MIC | MBC |
| Test organism | Resistance phenotype | (μg/ml) | (μg/ml) | (μg/ml) | (μg/ml) |
| Staphylococcus | ND | >250 | >250 | 7.8 | 31.25 |
| simulans ATCC 27848 | | | | | |
| Staphylococcus | ND | 250 | 250 | 7.8 | 15.62 |
| xylosus ATCC 29971 | | | | | |
| Staphylococcus cohnii | ND | 62.5 | 125 | 3.9 | 7.8 |
| ATCC 29974 | | | | | |
| Staphylococcus | ND | 250 | 250 | 3.9 | 7.8 |
| haemolyticus ATCC | | | | | |
| 29970 | | | | | |
| Staphylococcus | ND | >250 | >250 | 3.9 | 7.8 |
| warneri ATCC 27836 | | | | | |
| Staphylococcus capitis | ND | 250 | 250 | 1.95-3.9 | 3.9 |
| ATCC 27840 | | | | | |
| Staphylococcus | ND | 250 | 250 | 1.95 | 3.9 |
| hominis ATCC 27844 | | | | | |
| Staphylococcus | ND | 15.6 | 62.5 | 0.98 | 1.95 |
| auricularis ATCC | | | | | |
| 33753 | | | | | |
| Staphylococcus aureus | ND | 125 | 250 | 3.9 | 7.8 |
| ATCC 12600 | | | | | |
| S. aureus ATCC | ND | 250 | >250 | 3.9 | 7.8 |
| 12600-U | | | | | |
| S. aureus ATCC 12601 | ND | 250 | 250 | 7.8 | 15.6 |
| S. aureus ATCC 12602 | ND | 125 | 125 | 3.9 | 7.8 |
| S. aureus ATCC 12604 | ND | 125 | 250 | 7.8 | 7.8 |
| S. aureus ATCC 12605 | ND | 250 | 250 | 3.9 | 3.9 |
| S. aureus ATCC 12606 | ND | 250 | 250 | 7.8 | 7.8 |
| S. aureus ATCC 12607 | ND | 250 | 250 | 3.9 | 3.9 |
| S. aureus ATCC | ND | 250 | 250 | 7.8 | 15.6 |
| 29213 | | | | | |
| S. aureus ATCC | ND | 250 | 250 | 7.8 | 7.8 |
| 25923 | | | | | |
| S. aureus CPHL | Met/β-lactams* | 125 | 125-250 | 3.9 | 3.9 |
| EMRSA 15 | | | | | |
| S. aureus CPHL | Met/β-lactams* | 125 | 250 | 3.9 | 3.9 |
| EMRSA 16 | | | | | |
| S. aureus CPHL | Met/β-lactams* | 125 | 125-250 | 1.95 | 3.9 |
| EMRSA 17 | | | | | |
| S. aureus CPHL VISA | Van* (intermediate) | 125 | 125-250 | 3.9 | 7.8 |
| Mu3 | | | | | |
| S. aureus CPHL VISA | Van* (intermediate) | 125 | 250 | 3.9 | 7.8 |
| Mu50 | | | | | |
| S. aureus CPHL GISA | Van/Tec* (intermediate) | 125 | 250 | 7.8 | 15.6 |
| HO41340156 | | | | | |
| S. saprophyticus | ND | 250 | >250 | 3.9 | 7.8 |
| NCTC 7292 | | | | | |
| S. epidermidis (NCTC | ND | 250 | 250 | 3.9 | 7.8 |
| 11047 | | | | | |

[Abbreviations: American Type Culture Collection (ATCC), Central Public Health Laboratory UK (CPHL), National Collection of Type Cultures (NCTC), Methicillin (Met), Vancomycin (Van), Teicoplanin (Tec), not determined (ND), epidemic methicillin resistant S. aureus (EMRSA), vancomycin intermediate S. aureus (VISA), glycopeptide resistant S. aureus (GISA).]
*Other uncharacterised antibiotic resistances may be present.

TABLE 6

| | | | | SDDA | |
|---|---|---|---|---|---|
| | BP DDA | TBHQ | BP + TBHQ | increase | SDDA area |
| Test organism | (mm) | DDA (mm) | SDDA (mm) | (mm) | increase (%) |
| Staphylococcus | 9.94 | 53.07 | 65.32 (±1.26) | 12.25 | 51.49 |
| simulans ATCC 27848 | (±0.36) | (±0.83) | | | |
| Staphylococcus xylosus | 10.99 | 49.72 | 64.48 (±2.81) | 14.76 | 68.19 |
| ATCC 29971 | (±0.31) | (±0.48) | | | |
| Staphylococcus cohnii | 11.51 | 60.40 | 78.40 (±3.95) | 18.00 | 68.48 |
| ATCC 29974 | (±0.96) | (±3.95) | | | |
| Staphylococcus | 11.10 | 51.08 | 76.94 (±0.94) | 25.86 | 126.88 |
| haemolyticus ATCC | (±0.36) | (±1.73) | | | |
| 29970 | | | | | |
| Staphylococcus | 12.04 | 54.43 | 76.31 (±1.09) | 21.88 | 96.56 |
| warneri ATCC 27836 | (±0.48) | (±4.10) | | | |
| Staphylococcus capitis | 12.46 | 73.90 | 76.62 (±0.63) | 2.72 | 7.50 |
| ATCC 27840 | (±0.18) | (±2.67) | | | |
| Staphylococcus | 11.83 | 64.59 | 77.15 (±1.61) | 12.56 | 42.67 |
| hominis ATCC 27844 | (±0.18) | (±1.42) | | | |
| Staphylococcus | 21.56 | 80.18 | 77.77 (±1.48) | −2.41 | −5.92 |
| auricularis ATCC | (±0.96) | (±0.48) | | | |
| 33753 | | | | | |
| Staphylococcus aureus | 11.99 | 49.24 | 69.55 (±1.79) | 20.31 | 99.51 |
| ATCC 12600 | (±0.0) | (±3.52) | | | |
| S. aureus ATCC | 12.20 | 51.24 | 70.91 (±2.57) | 19.67 | 91.51 |
| 12600-U | (±0.66) | (±2.15) | | | |
| S. aureus ATCC 12601 | 11.68 | 57.34 | 73.23 (±0.79) | 15.89 | 63.10 |
| | (±0.0) | (±0.79) | | | |
| S. aureus ATCC 12602 | 12.73 | 57.66 | 73.23 (±0.55) | 15.57 | 61.30 |
| | (±0.18) | (±3.82) | | | |
| S. aureus ATCC 12604 | 12.52 | 55.03 | 72.07 (±3.40) | 17.04 | 71.52 |
| | (±0.18) | (±2.68) | | | |
| S. aureus ATCC 12605 | 12.20 | 59.55 | 77.65 (±0.95) | 18.10 | 70.03 |
| | (±0.48) | (±1.42) | | | |
| S. aureus ATCC 12606 | 11.68 | 53.34 | 56.92 (±0.66) | 3.58 | 13.87 |
| | (±0.32) | (±1.67) | | | |
| S. aureus ATCC 12607 | 12.31 | 54.50 | 72.49 (±2.39) | 17.99 | 76.91 |
| | (±0.32) | (±1.82) | | | |
| S. aureus ATCC | 9.61 | 46.79 | 66.11 (1.96) | 19.32 | 99.63 |
| 29213 | (±0.18) | (±1.30) | | | |
| S. aureus ATCC | 10.86 | 44.70 | 62.56 (±1.19) | 17.86 | 95.87 |
| 25923 | (±0.36) | (±0.90) | | | |
| S. aureus CPHL | 10.17 | 55.14 | 72.85 (±0.96) | 17.72 | 74.55 |
| EMRSA 15 | (±0.18) | (±1.49) | | | |
| S. aureus CPHL | 10.80 | 67.72 | 73.27 (±1.44) | 5.56 | 17.06 |
| EMRSA 16 | (±0.18) | (±3.10) | | | |
| S. aureus CPHL | 11.74 | 51.05 | 67.82 (±0.48) | 16.77 | 76.49 |
| EMRSA 17 | (±0.36) | (±0.48) | | | |
| S. aureus CPHL VISA | 10.90 | 50.73 | 61.53 (±1.61) | 10.80 | 47.11 |
| Mu3 | (±0.36) | (±1.49) | | | |
| S. aureus CPHL VISA | 11.64 | 50.11 | 63.63 (±1.27) | 13.52 | 61.24 |
| Mu50 | (±0.63) | (±1.55) | | | |
| S. aureus CPHL GISA | 12.16 | 75.37 | 75.26 (±2.82) | −0.11 | 0.00 |
| HO41340156 | (±0.18) | (±2.14) | | | |
| S. saprophyticus | 11.38 | 41.67 | 60.47 (±1.13) | 18.80 | 110.59 |
| NCTC 7292 | (±0.36) | (±1.13) | | | |
| S. epidermidis (NCTC | 10.65 | 57.34 | 76.55 (±0.48) | 19.21 | 78.23 |
| 11047 | (±0.63) | (±0.83) | | | |

Taking an increase in zone size of greater than 5 mm as an indicator of potential synergy, for most of the staphylococcal strains tested the combination of peroxide and hydroquinone exhibits a potentially synergistic antibacterial interaction. Even in cases where the SDDA zone increase is less than 5 mm, the interaction appears to be indifferent rather than strongly antagonistic, thus providing the opportunity to prepare antimicrobial formulations with reduced levels of the potentially irritant peroxide yet without undue loss of antimicrobial activity. These results are likely to be of particular clinical value for the antibiotic resistant test strains.

### EXAMPLE 6

### Activity Against P. acnes—MIC, MBC & (S)DDA Assays

The following experiments all used P. acnes NCTC 737 as the test organism.

MIC, MBC and DDA assays, as described above, were carried out using BP and a range of different benzoquinones and hydroquinones. Supplemented DDA assays, in the presence of salt and lipid, were also conducted.

Each of the quinones was then subjected, in combination with BP, to the SDDA assay described above. In each case, increases in zone diameter (mm) and area (%) were measured with respect to those observed for the compound showing the larger zone diameters during the previous disc diffusion assays on the individual compounds.

For all (S)DDA assays, 200 μg of each compound was loaded onto each disc. The solvents used were DMSO (for BP, 2,3-dimethyl-p-hydroquinone and 2-ethyl-p-hydroquinone) and ethanol (for TBHQ, thymoquinone, p-hydroquinone and p-benzoquinone).

The MIC, MBC and DDA results are shown in Table 7 below and the SDDA results in Table 8. All results are collated from a number of experiments.

TABLE 7

| | | | | DDA + | DDA + |
|---|---|---|---|---|---|
| | MIC | MBC | DDA | salt | lipid |
| Test compound | (μg/ml) | (μg/ml) | (mm) | (mm) | (mm) |
| BP | 31.25 | 62.5 | 24.25 | n/a | n/a |
| | | | (±2.18) | | |
| TBHQ | 7.8 | 15.6 | 9.95 | 19.48 | 10.37 |
| | | | (±0.31) | | |
| thymoquinone | 15.6 | 31.25 | 27.46 | 46.49 | 49.01 |
| | | | (±1.18) | | |
| p-hydroquinone | >250 | >250 | 0.0 | 0.0 | 0.0 |
| | | | (±0.0) | | |
| p-benzoquinone | 62.5 | 62.5 | 24.87 | 31.44 | 37.98 |
| | | | (±0.82) | | |
| 2,3-dimethyl-p- | 7.8 | 15.6 | 18.75 | 14.38 | 13.75 |
| hydroquinone | | | (±1.65) | | |
| 2-ethyl-p-hydroquinone | 7.8 | 31.25 | 9.90 | 9.38 | 0.0 |
| | | | (±0.36) | | |

TABLE 8

| | SDDA | SDDA | |
|---|---|---|---|
| | with BP | increase | SDDA area |
| Test quinone | (mm) | (mm) | increase (%) |
| TBHQ | 42.80 | 18.55 | 211.51 |
| | (±0.90) | | |
| thymoquinone | 36.79 | 9.33 | 79.46 |
| | (±0.36) | | |
| p-hydroquinone | 33.16 | 8.91 | 87.01 |
| | (±1.47) | | |
| p-benzoquinone | 33.16 | 8.29 | 77.78 |
| | (±0.72) | | |
| 2,3-dimethyl-p-hydroquinone | 33.65 | 10.63 | 113.68 |
| | (±1.26) | | |
| 2-ethyl-p-hydroquinone | 31.25 | 8.23 | 84.28 |
| | (±0.0) | | |

The data in Tables 7 and 8 show that each of the quinones alone is active against P. acres NCTC 737, some (in particular TBHQ, 2,3-dimethyl-p-hydroquinone and 2-ethyl-p-hydroquinone) strongly so. BP is also active against the organism, although less strongly than the more active quinones. In most cases quinone activity is maintained, at least to some extent, in the presence of salt and lipid, which are important constituents of the human skin environment. In some cases quinone activity appears to be potentiated by one or both of the supplements.

Again when BP is combined with a benzo/hydroquinone, the SDDA data imply a potentially synergistic antimicrobial interaction between the two, in each case with a significant increase in zone diameter over that exhibited by either compound alone.

The BP/TBHQ SDDA assay was repeated in the presence of salt and lipid, as described above. Antibacterial synergy appeared to be retained under these supplemented conditions, the zone diameter increase being 18.34 mm and the area increase 419.8%.

### EXAMPLE 7

### Activity Against Propionibacteria—FIC Assays

Mixtures of BP and TBHQ, containing various relative proportions of the two actives, were then subjected to FIC assays as described above. The test organism used was the in-house P. granulosum strain PRP-055, as described above. The solvents used were DMSO for the BP and ethanol for the TBHQ.

An enhanced antimicrobial activity (lowest FICI value 0.53) was observed as a result of combining the peroxide with the hydroquinone. A representative isobologram is shown in FIG. 2.

### EXAMPLE 8

### Activity Against Other Propionibacteria—MIC, MBC & (S)DDA Assays

The activities of BP, TBHQ and combinations of the two were tested against other Propionibacterium spp strains, including some with known antibiotic resistance. MIC, MBC and (S)DDA assays were carried out as described above for each of the strains.

For all (S)DDA assays, 200 μg of each compound was loaded onto each disc. The solvents used were DMSO for the BP and ethanol for the TBHQ.

The MIC and MIC results are shown in Table 9 below and the (S)DDA results in Table 10. All results are collated from a number of experiments. Table 9 indicates the resistance phenotype for each of the test strains.

TABLE 9

| | | | | TBHQ | TBHQ |
|---|---|---|---|---|---|
| | Resistance | BP MIC | BP MBC | MIC | MBC |
| Test organism | phenotype | (μg/ml) | (μg/ml) | (μg/ml) | (μg/ml) |
| P. acnes NCTC 737 | None | 15.6 | 31.25 | 7.8 | 15.6 |
| P. granulosum | None | 15.6 | 15.6 | 3.9 | 7.8 |
| NCTC 11865 | | | | | |
| P. acnes PRP-002 | Tet/MLS | 15.6 | 31.25 | 3.9 | 7.8 |
| P. acnes PRP-003 | Tet | 31.25 | 31.25 | 7.8 | 7.8 |
| P. acnes PRP-004 | Tet | 7.8 | 15.6 | 1.95 | 7.8 |
| P. granulosum | MLSK | 15.6 | 15.6 | 62.5 | 62.5 |
| PRP-005 | | | | | |
| P. acnes PRP-007 | Clin | 31.25 | 62.5 | 3.9 | 7.8 |
| P. acnes PRP-008 | Clin | 31.25 | 31.25 | 3.9 | 7.8 |
| P. acnes PRP-010 | MLSK | 31.25 | 31.25 | 3.9 | 15.6 |
| P. acnes PRP-017 | MLS | 31.25 | 62.5 | 3.9 | 7.8 |
| P. acnes PRP-023 | MLSK | 31.25 | 62.5 | 3.9 | 7.8 |
| P. acnes PRP-026 | MLS | 15.6 | 31.25 | 3.9 | 7.8 |
| P. granulosum | MLS | 62.5 | 62.5 | 15.6 | 15.6 |
| PRP-043 | | | | | |
| P. granulosum | MLS | 31.25 | 31.25 | 15.6 | 31.25 |
| PRP-044 | | | | | |
| P. acnes PRP-046 | None | 31.25 | 62.5 | 1.95 | 7.8 |
| P. acnes PRP-053 | Tet/MLS | 31.25 | 62.5 | 3.9 | 7.8 |
| P. granulosum | None | 15.6 | 15.6 | 3.9 | 7.8 |
| PRP-055 | | | | | |
| P. acnes PRP-059 | MLS | 62.5 | 125 | 3.9 | 7.8 |
| P. acnes PRP-068 | Ery | 62.5 | 125 | 3.9 | 7.8 |
| P. acnes PRP-101 | Tet/MLS | 15.6 | 31.25 | 3.9 | 7.8 |
| P. acnes PRP-102 | Tet/MLS | 62.5 | 62.5 | 7.8 | 15.6 |
| P. avidum | None | 31.25 | 62.5 | 3.9 | 3.9 |
| ATCC 25577 | | | | | |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]

TABLE 10

| | | | | SDDA | |
|---|---|---|---|---|---|
| | BP DDA | TBHQ DDA | BP + TBHQ | increase | SDDA area |
| Test organism | (mm) | (mm) | SDDA (mm) | (mm) | increase (%) |
| P. acnes NCTC 737 | 19.25 (±0.18) | 8.65 (±0.31) | 35.51 (±0.00) | 16.26 | 240.28 |
| P. granulosum NCTC 11865 | 24.19 (±0.94) | 11.00 (±0.00) | 35.64 (±0.18) | 11.45 | 117.07 |
| P. acnes PRP-002 | 25.69 (±0.66) | 28.42 (±0.95) | 40.56 (±1.28) | 12.14 | 103.34 |
| P. acnes PRP-003 | 25.69 (±0.66) | 38.01 (±1.02) | 47.70 (±0.00) | 9.69 | 57.48 |
| P. acnes PRP-004 | 31.17 (±1.02) | 30.32 (±1.97) | 48.85 (±3.10) | 17.68 | 145.62 |
| P. granulosum PRP-005 | 22.95 (±0.64) | 0.00 (±0.00) | 25.73 (±0.18) | 2.78 | 25.69 |
| P. acnes PRP-007 | 23.88 (±1.42) | 13.43 (±2.49) | 39.38 (±0.82) | 15.5 | 171.95 |
| P. acnes PRP-008 | 27.80 (±1.29) | 14.47 (±0.90) | 42.89 (±0.65) | 15.09 | 138.03 |
| P. acnes PRP-010 | 30.39 (±0.82) | 18.71 (±0.18) | 41.76 (±3.11) | 11.37 | 89.07 |
| P. acnes PRP-017 | 23.28 (±0.48) | 18.40 (±1.08) | 53.73 (±0.36) | 30.45 | 432.68 |
| P. acnes PRP-023 | 24.11 (±0.65) | 23.90 (±0.00) | 63.08 (±2.40) | 38.97 | 584.52 |
| P. acnes PRP-026 | 26.52 (±0.65) | 8.18 (±0.72) | 41.53 (±2.59) | 15.01 | 145.23 |
| P. granulosum PRP-043 | 22.48 (±1.19) | 10.56 (±0.48) | 25.72 (±0.63) | 3.24 | 30.9 |
| P. granulosum PRP-044 | 29.31 (±0.54) | 10.70 (±0.65) | 30.76 (±1.47) | 1.45 | 10.14 |
| P. acnes PRP-046 | 24.36 (±0.36) | 17.46 (±1.58) | 50.19 (±0.54) | 25.83 | 324.5 |
| P. acnes PRP-053 | 29.72 (±0.48) | 23.49 (±1.44) | 51.65 (±1.41) | 21.93 | 202.03 |
| P. granulosum PRP-055 | 21.51 (±0.47) | 13.07 (±0.78) | 41.17 (±0.99) | 19.66 | 266.34 |
| P. acnes PRP-059 | 23.49 (±0.48) | 17.56 (±0.72) | 59.86 (±1.25) | 36.37 | 549.39 |
| P. acnes PRP-068 | 28.79 (±0.95) | 20.89 (±1.08) | 69.94 (±0.95) | 41.15 | 490.16 |
| P. acnes PRP-101 | 25.73 (±1.99) | 0.0 (±0.00) | 37.77 (±1.89) | 12.04 | 115.48 |
| P. acnes PRP-102 | 29.62 (±1.12) | 23.80 (±0.10) | 51.13 (±0.54) | 21.51 | 197.98 |
| P. avidum ATCC 25577 | 25.83 (±0.95) | 9.16 (±0.48) | 40.93 (±1.08) | 15.1 | 151.09 |

Taking an increase in zone size of greater than 5 mm as an indicator of potential synergy, for most of the propionibacterial strains tested the combination of peroxide and hydroquinone exhibits a potentially synergistic antibacterial interaction. In some cases only a small increase in zone diameter is seen in the SDDA tests, potentially indicative of an indifferent interaction between the two test compounds—this could still provide the opportunity to prepare antimicrobial formulations with reduced levels of the potentially irritant peroxide yet without undue loss of antimicrobial activity.

These results are likely to be of particular clinical value for the antibiotic resistant test strains.

### EXAMPLE 9

### Activity Against P. acnes—Other Peroxides

Four other peroxides were subjected to DDA assays against P. acnes NCTC 737, including in combination with TBHQ. The results are shown in Table 11 below, each being an average (mean) of three replicate tests.

Again for all (S)DDA assays, 200 μg of each compound was loaded onto each disc. The solvents used were dH₂O for the metal peroxides, and ethanol for the TBHQ and t-butyl hydroperoxide.

TABLE 11

| | | SDDA | | SDDA |
|---|---|---|---|---|
| | | with | SDDA | area |
| | DDA | TBHQ | increase | increase |
| Test compound | (mm) | (mm) | (mm) | (%) |
| TBHQ | 15.0 | N/A | N/A | N/A |
| | (±1.19) | | | |
| calcium peroxide | 0.0 | 38.08 | 23.08 | 544.48 |
| | (±0.00) | (±0.83) | | |
| magnesium peroxide | 0.0 | 37.45 | 22.45 | 523.33 |
| | (±0.00) | (±0.94) | | |
| sodium peroxide | 0.0 | 22.66 | 7.66 | 128.21 |
| | (±0.00) | (±1.37) | | |
| t-butyl hydroperoxide | 0.0 | 25.49 | 10.49 | 188.77 |
| | (±0.00) | (±0.31) | | |

The Table 11 data indicate that peroxides other than benzoyl peroxide can exhibit a synergistic antimicrobial effect when combined with a benzo/hydroquinone such as TBHQ. In each case the activity of the combination, against P. acnes NCTC 737, was significantly higher than that of either of the individual test compounds alone.

The individual MICs and MBCs for the three metal peroxides against P. acnes NCTC 737 were all greater than 250 μg/ml. It is thus likely that combining such a peroxide with a suitable benzo/hydroquinone could allow the use of significantly lower peroxide concentrations than might otherwise be necessary to ensure an antimicrobial effect.

### EXAMPLE 10

### Activity Against E. faecalis

The activities of BP, TBHQ and combinations of the two were tested against E faecalis ATCC 29212, using MIC, MBC and (S)DDA assays as described above.

For the (S)DDA assays, 200 μg of each compound was loaded onto each disc. The solvents used were DMSO for the BP and ethanol for the TBHQ.

The MIC and MBC results are shown in Table 12 below and the (S)DDA results in Table 13. All results are collated from a number of experiments.

TABLE 12

| | MIC | MBC |
|---|---|---|
| Test compound | (μg/ml) | (μg/ml) |
| BP | >250 | >250 |
| TBHQ | 15.6 | 31.25 |

TABLE 13

| | | | SDDA | |
|---|---|---|---|---|
| | | BP + TBHQ | increase | SDDA area |
| Test compound | DDA (mm) | SDDA (mm) | (mm) | increase (%) |
| BP | 0.0 | 55.33 | 21.66 | 170.04 |
| | (±0.0) | (±2.31) | | |
| TBHQ | 33.67 | — | — | — |
| | (±0.58) | | | |

Tables 12 and 13 show that formulations according to the invention have activity against E. faecalis as well as against a range of different staphylococcal and propionibacterial strains as shown in Examples 5 and 8. Again, although the peroxide alone has relatively low activity against this organism, when combined with the hydroquinone a significant level of antibacterial synergy is observed.

BP/TBHQ combinations were also subjected to DDA and SDDA tests against Acinetobacter baumanni ATCC 19606, Escherichia coli ATCC 25922, Haemophilus influenzae ATCC 49247, Klebsiella pneumoniae ATCC 700603, Pseudomonas aeruginosa ATCC 27853 and Streptococcus pyogenes ATCC 12344. Both test compounds were used at 200 μg per disc, the BP in DMSO and the TBHQ in ethanol. In these cases the SDDA data failed to give a clear indication of a synergistic interaction.

### EXAMPLE 11

### Topical Anti-Acne Formulations

The results from Examples 1 to 10 show that the combination of a peroxide and a benzo/hydroquinone can be an effective antimicrobial agent, in particular against the bacteria associated with skin infections, in many cases with a synergistic impact on the antimicrobial activity of the combination compared to that of the individual compounds alone. This can be of use in preparing antimicrobial formulations, in particular for topical application to the skin, for either prophylactic or therapeutic use in any context where such bacteria are thought to be involved as possible sources of infection.

Even in cases where the combination has an additive (indifferent), as opposed to synergistic, antimicrobial activity compared to that of the individual compounds, this can still be of considerable benefit when preparing formulations for topical use. A benzo/hydroquinone may be used to replace a proportion of a peroxide, such as benzoyl peroxide, thus lowering the irritant effect of the combination without undue loss of antimicrobial activity. This maintenance of, or in cases improvement in, antimicrobial activity could not necessarily have been predicted from the known activities and uses of peroxides and benzo/hydroquinones individually.

A topical formulation for use in treating acne may for example be prepared by combining a peroxide such as benzoyl peroxide (or any of those tested in Example 9) with a suitable benzoquinone or hydroquinone, in particular an alkyl-substituted benzo/hydroquinone such as TBHQ, in a suitable fluid vehicle and optionally together with conventional additives. Such vehicles and additives may be for instance as found in Williams' “Transdermal and Topical Drug Delivery”, Pharmaceutical Press, 2003 and other similar reference books, and/or in Rolland A et al, “Site-specific drug delivery to pilosebaceous structures using polymeric microspheres”, Pharm. Res. 1993; 10: 1738-44; Mordon S et al, “Site-specific methylene blue delivery to pilosebaceous structures using highly porous nylon microspheres: an experimental evaluation”, Lasers Surg. Med. 2003; 33: 119-25; and Alvarez-Roman R et al, “Skin penetration and distribution of polymeric nanoparticles”, J. Controlled Release 2004; 99: 53-62.

The formulation may be prepared and administered using known techniques. It may for example take the form of a cream, lotion or gel. It may be applied to infected areas of the skin, and/or to areas susceptible to future infection, with a frequency dependent on the nature and severity of the condition and the concentration of the peroxide, quinone and any other active agents in the formulation, for instance on a daily or twice daily basis.

The concentrations of the peroxide and the benzo/hydroquinone may be in the ranges described above, and will be determined based on the intended use of the formulation, its intended mode of administration and the activities of the particular chosen active agents.

### EXAMPLE 12

### Topical Formulation for Use Against Staphylococcal Infections

A formulation for use against S. aureus or other staphylococci may be prepared by combining a peroxide such as benzoyl peroxide with a benzo/hydroquinone such as TBHQ in a similar manner to that described for the anti-acne formulation. The ingredients may in this case be formulated as a spray, for instance for application to work surfaces or surgical instruments; as a cleansing gel or lotion for instance for hand washing; as a nasal spray for application to the anterior nares or in many other appropriate forms. Such a formulation may in particular be used prophylactically, eg, to reduce the risk of outbreaks of MRSA or similar infections.

1. An antimicrobial formulation containing (a) a peroxide selected from the group consisting of diacyl peroxides and metal peroxides and (b) a para-benzoquinone or para-hydroquinone which is substituted with one or more groups selected from alkyl, alkoxy, halogen, hydroxyl, nitro (—NO₂) and amine (—NR₂, where each R is independently either hydrogen or hydrocarbyl) groups attached to carbon atoms in the cyclohexadiene ring, wherein the concentration of the peroxide is up to 2.5% w/v.

2. A formulation according to claim 1, which is suitable for topical application to the skin or nares.

3-9. (canceled)

10. A formulation according to claim 1, wherein the diacyl peroxide is benzoyl peroxide.

11-17. (canceled)

18. A formulation according to claim 1, wherein the substituents on the benzo/hydroquinone are selected from alkyl groups.

19-28. (canceled)

29. A formulation according to claim 18, wherein the benzo/hydroquinone is TBHQ.

30-64. (canceled)

65. A method for controlling the growth of a micro-organism, the method comprising applying, to an area infected or suspected to be infected or capable of becoming infected with the micro-organism, a combination of (a) a peroxide selected from the group consisting of diacyl peroxides and metal peroxides and (b) a para-benzoquinone or para-hydroquinone which is substituted with one or more groups selected from alkyl, alkoxy, halogen, hydroxyl, nitro (—NO₂) and amine (—NR₂, where each R is independently either hydrogen or hydrocarbyl) groups attached to carbon atoms in the cyclohexadiene ring.

66-67. (canceled)

68. A method according to claim 65, wherein the area to which the peroxide and the benzo/hydroquinone are applied is a non-living surface.

69-71. (canceled)

72. A method of increasing the antimicrobial activity of an antimicrobial formulation containing a peroxide selected from the group consisting of diacyl peroxides and metal peroxides, which method involves incorporating into the formulation a para-benzoquinone or para-hydroquinone which is substituted with one or more groups selected from alkyl, alkoxy halogen, hydroxyl, nitro (—NO₂) and amine (—NR₂, where each R is independently either hydrogen or hydrocarbyl) groups attached to carbon atoms in the cyclohexadiene ring.

73. Method according to claim 72, which is for the purpose of reducing the amount of the peroxide in the formulation, and/or reducing the skin irritancy or another undesired property of the formulation, without undue loss of antimicrobial activity.

74-75. (canceled)

76. Method for the treatment, in a human or animal patient, of a condition which is either caused, exacerbated or transmitted by microbial activity, the method involving the administration to the patient of a pharmaceutically or veterinarily effective amount of (a) a peroxide selected from the group consisting of diacyl peroxides and metal peroxides and (b) a para-benzoquinone or para-hydroquinone which is substituted with one or more groups selected from alkyl, alkoxy, halogen, hydroxyl, nitro (—NO₂) and amine (—NR₂, where each R is independently either hydrogen or hydrocarbyl) groups attached to carbon atoms in the cyclohexadiene ring.

77. Method according to claim 76, wherein the condition is caused, exacerbated or transmitted by staphylococcal and/or propionibacterial activity.

78. Method according to claim 76, wherein the condition is a skin or skin structure condition.

79. Method according to claim 78, wherein the condition is acne.

80. Method according to claim 76, wherein the condition is a staphylococcal infection.
